(19) United States
(12) Patent Application Publication
(10) Pub. No.: US 2019/0254961 A1
(43) Pub. Date: Aug. 22, 2019

(54) Hemostatic Nose Plugs

(71) Applicant: Rhinoclear Nasal Care Solutions Inc., Calgary (CA)

(72) Inventors: Bradford Mechor, Calgary (CA); Merle Olson, Calgary (CA)

(73) Assignee: Rhinoclear Nasal Care Solutions Inc., Calgary, AB (CA)

(21) Appl. No.: 16/345,452

(22) PCT Filed: Oct. 27, 2017
(86) PCT No.: PCT/CA2017/051285

§ 371 Date: Apr. 26, 2019

Related U.S. Application Data
(60) Provisional application No. 62/413,527, filed on Oct. 27, 2016.

(51) Int. Cl. A61K 9/00; A61F 13/20; A61K 33/26; A61K 45/06

(52) U.S. Cl. CPC A61K 9/0043; A61K 45/06; A61K 33/26; A61F 13/2005

(57) ABSTRACT

A deformable nose plug, comprising a deformable core material and an outer coating provided on said deformable core material, wherein said said outer coating comprises a hemostatic compound. The nose plug has a tapered distal end for insertion into a nostril and a proximal end whereby the nose plug is inserted into the nostril. The deformable core material may comprise a multi-phasic core material wherein a first phase of the multi-phasic core material is characterized by a solid or a semi-solid structure at a temperature of 35° C. or less, and wherein a second phase of the multi-phasic core material is characterized by liquifaction at a temperature of 37° C. or greater. Alternatively, the deformable core material may comprise a porous resiliently compressible substrate. Alternatively, the deformable porous resiliently compressible core material may be one of polymeric sponges, naturally occurring sponges, fluffy cellulosic materials, cotton, rolled gauze, and mixtures thereof.

### TECHNICAL FIELD

This disclosure generally relates to a treatment for nosebleeds. In particular, the disclosure relates to hemostatic-coated nose plugs that stop bleeding on contact with nasal tissues.

### BACKGROUND

Control of nosebleeds is a challenge and a common problem. Nosebleeds often result from a physical trauma or injury. However, nosebleeds can occur unexpectedly as a result of sinus tissue infections of the sinus tissue, excessive drying of the nasal mucous membranes, and high blood pressure. In many cases, the capillary vessels in nasal passages may become weakened or scarred as a result of prolonged infections or physical perturbations and will rupture unexpectedly during periods of physical or emotional stress or during physical exertion. Such weakened capillaries are not able to completely heal between nosebleed episodes. Regardless of the cause of nosebleeds, their occurrence is usually unpredictable, very inconvenient, and difficult to stop because of suitable materials are not readily available or at hand.

There are currently many products available for stopping nosebleeds but very few have practical ease of use. Such products include sponges, gauzes or cotton pads that may be inserted into the nose. Such products are not practical and are used infrequently because they do not have high degrees of absorptive capacity and therefore must be removed and replaced a number of times before a nosebleed is completely stopped. Furthermore, such products frequently cause debridement of the healing tissue inside the nose during their insertion into and removal from nostrils causing discomfort and further bleeding. In addition, such products must be retained in the nostrils for extended periods of time after insertion, which may cause predispose the occurrence of nasal infections.

### SUMMARY

The present disclosure relates to a hemostatic nose plugs for insertion into a mammalian nose to stop bleeding. According to one aspect, the cylindrical nose plugs comprises an inner core and an outer coating. According to another aspect, the nose plugs comprises an inner core that is a semi-solid gel matrix that liquefies at body temperature and an outer coating comprising a hemostatic substance. According to another aspect, an insertion and removal component is integrally associated with the inner core.

### BRIEF DESCRIPTION OF THE DRAWINGS

The embodiments of the present disclosure will be described with reference to the following drawings in which:

FIG. 1A is a side view of the nose plug according to one embodiment of the present disclosure, and FIG. 1B is a cross-sectional view of the nose plug;

FIG. 2 is a cross -sectional side view of another embodiment of another nose plug according to of the present disclosure; and

FIG. 3 is a cross-sectional side view of another embodiment of another nose plug according to the present disclosure.

### DETAILED DESCRIPTION

The embodiments of the present disclosure generally pertain to nose plugs having hemostatic coatings thereon for affecting immediate cessation of nosebleeds upon insertion of the nose plugs into nasal cavities. According to one aspect, the present nose plugs comprise a core material for receiving and supporting thereon a coating comprising a compound with hemostatic properties. According to another aspect, each of the present nose plugs may additionally comprise an applicator stick engaged with the core material. Alternatively, each of the present nose plugs may comprise a pliable material engaged with the core material wherein a portion of the pliable material extends outward from the core material.

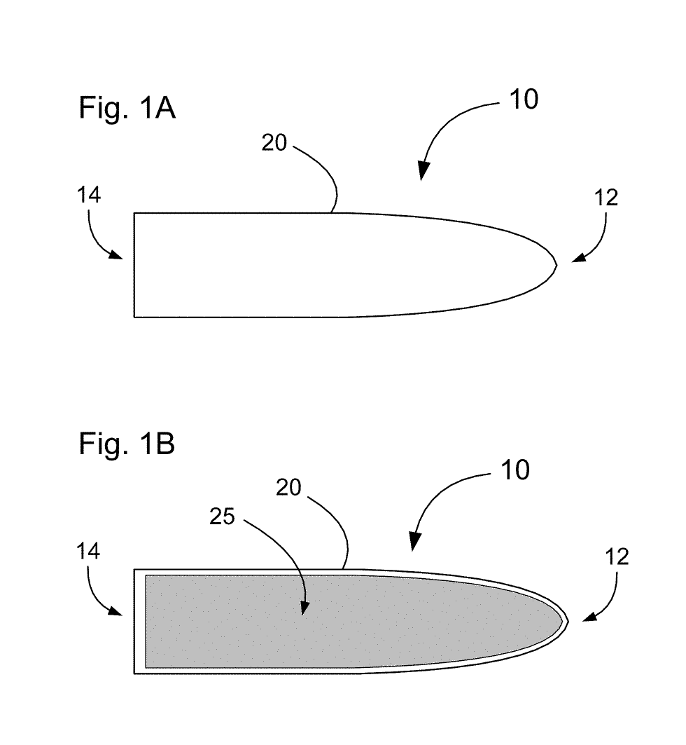

An embodiment of a nose plug 10 according to the present disclosure is shown in FIGS. 1A and 1B and comprises a multi-phasic core material 25 that has been coated with a hemostatic composition 20.

A suitable multi-phasic core material 25 may be formed from a mixture of materials that are solids or semi-solids at temperatures lower than about 36° C. (i.e., a first phase) and that liquify at a temperature of 37° C. or higher (i.e., a second phase). Examples of suitable mixtures of materials for preparing the multi-phasic core material 25 include of two or more of cocoa butter, polyethylene glycols, hydrogels, glycerin, polyglycolysed glycerides, and glycerinated gelatin. Suitable polyglycolysed glycerides include but are not limited to arachidic acid, lauric acid, myristic acid, aleic acid, palmitic acid, and steric acid. The core material 25 may additionally comprise an excipient exemplified by monodecanoyl-glycerol, monolauroyl-glycerol, fatty acid triglycerides, thylene glycol, triethylene glycol, propylene glycol, butylene glycols, 1,2-propylene glycol, and the like. The multi-phasic core material 25 may additionally comprise a lubricant exemplified by mineral oil, vegetable oil, polyisobutene, polyalkyleglycol, polyolefin, diester, polymerester, vegetable stearin, magnesium stearate, hydrogenated vegetable oils, cocoa butter, theobroma oil, hydrogenated corn oil, palm oil, palm kernel oil, coconut oil, stearic acid, and the like. The multi-phasic core material 25 may additionally comprise an emollient exemplified by salicylic acid, ammonium lactate, urea, dimenticone, and the like.

The multi-phasic core material may be prepared by mixing together the selected materials in heated water, for example, at a temperature from a range of 40° C. to 90° C. and anywhere therebetween, and then pouring the mixture into molds for curing and forming. One example of a suitable mixture of material for the multi-phasic core is about 14% gelatin plus about 70% glycerol plus about 16% water. Another example of a suitable mixture is about 33% PEG 4000 plus about 47% PEG 6000 plus about 20% water. Suitable molds for forming the multi-phasic core material into nose plugs include metal molds, flexible rubber molds, silcone molds, and the like. Each nose plug of the present disclosure generally comprises about 0.5 g, about 0.6 g, about 0.7 g, about 0.8 g, about 0.9 g, about 1.0 g, about 1.1 g, about 1.2 g, about 1.3 g, about 1.4 g, about 1.5 g of material and therebetween. Particularly suitable is about 1.0 g of core material.

The nose plugs of the present disclosure generally have an elongate cylindrical shape with a tapered conical distal end 12 (i.e., the end that is inserted into a nostril). Such shapes are commonly referred to as bullet-shaped or torpedo-shaped. It is optional for the distal end 12 of the nose plugs 10 to have a frusticonical taper terminating with a rounded end. The proximal end 14 of a nose plug 10 of the present disclosure, may be flat. Alternatively, the proximal end of the nose plug may be concave. Alternatively, the proximal end of the nose plug may be convex. The diameter of the multi-phasic core material of the nose plugs 10 may be selected from a range of about 0.5 cm to about 1.75 cm and therebetween. The diameter of a nose plug 10 approximate its proximal end 14 may be same as the diameter of the central body portion of the nose plug 10, i.e., the nose plug 10 may have the same diameter from its proximal end 14 until the onset of tapering to its distal end 12. Alternatively, the diameter of a nose plug according to the present disclosure, may be slightly flared outward approximate its proximal end. Alternatively, the diameter of a nose plug according to the present disclosure, may be slightly flared outward approximate its proximal end. The length of the multi-phasic core material of the nose plugs may be selected from a range of about 1.5 cm to about 3.5 cm and therebetween. The multi-phasic nature of the core material makes it deformable as the present nose plug is inserted into a nostril so that a large portion of the outer covering of the nose plug is in at least partial and preferably full contact with the nasal membranes lining the nostril.

After the multi-phasic cores of the nose plugs have been formed and solidified, a solution comprising one or more hemostatic compounds is coated onto the outer surface of the nose plug cores, for example, by dipping or by spraying or other suitable coating processes, after which the coated nasal plugs are dried. Suitable hemostatic compounds include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof.

For use to stop a nosebleed, the present nose plug may be taken out of its container and simply inserted into the nostril such that some or all of the elongate outer surface of the nose plug is in contact with the nasal tissues lining the nostril and/or nasal passage. The hemostatic compound in the outer coating of the nose plug will cause cessation of bleeding at a capillary source(s) within the nostril and/or nasal passage. Additionally, because the of the deformable nature of the multi-phasic core material and the resiliently compressible substrate, the present nose plugs will provide a compression pressure onto and about the bleeding areas within the nostril and nasal passage. After insertion of the present nose plug into a nostril, the core material will slowly liquify as the nose plug is warmed to body temperature (i.e., 37° C.) and will slowly flow to the back of the nasal cavity, toward the pharynx from where it may be spit out, or alternatively, pass into the esophagus and moved to the stomach where it will digested. Once the nosebleed has stopped, the nose plug can be removed simply by blowing the nose.

It is within the scope of the present disclosure to incorporate components into the multi-phasic core material if so desired. Suitable components that may be incorporated including clotting factors such as thrombin, prothrombin, thromboplastin, fibrinogen, and the like; antiobiotic compositions such as penicillin, cephalosporin, tetracycline, macrolides, and the like; antimicrobial agents such as thymol, benzoic acid, phenolic acid, sorbic acids, alcohols, benzethonium chloride, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, propylparaben, sodium propionate, thimerosalaminoclycosides, glycopepetides, macrocodes, quinolones, streptogramins, carbapenems, and the like;

antihistamines such as chlorpheniramine, clemastine, brompheniramine, diphenhydramine, loratadine, cetirizine, fexofenadine, and the like; decongestants such as pseudoephedrine, phenylephrine, oxymetazoline and the like; and combinations thereof.

Other suitable components for incorporation into the multi-phasic core material include emollients exemplified by mineral oil, mixtures of mineral oil and lanolin alcohols, cetyl alcohol, cetostearyl alcohol, petrolatum, petrolatum and lanolin alcohols, cetyl esters wax, cholesterol, glycerin, glyceryl monostearate, isopropyl myristate, isopropyl palmitate, lecithin, allyl caproate, althea officinalis extract, arachidyl alcohol, argobase EUC, butylene glycol, dicaprylate/dicaprate, acacia, allantoin, carrageenan, cetyl dimethicone, cyclome hicone, diethyl succinate, dihydroabietyl behenate, dioctyl adipate, ethyl laurate, ethyl palmitate, ethyl stearate, isoamyl laurate, octanoate, PEG-75, lanolin, sorbitan laurate, walnut oil, wheat germ oil, super refined almond, super refined sesame, super refined soyabean, octyl palmitate, caprylic/capric triglyceride, glyceryl cocoate, and the like. An emollient, if present, is present in the compositions described herein in an amount by weight of the composition of about 1% to about 30%, about 3% to about 25%, or about 5% to about 15%. Illustratively, one or more emollients are present in a total amount of about 1% by weight, about 2%, about 3%, about 4%, about 5%, about 6%, about 7%, about 8%, about 9%, about 10%, about 1 1%, about 12%, about 13%, about 14%, about 1%, about 16%, about 17%, about 18%, about 19%, about 20%, about 21%, about 22%, about 23%, about 24%, about 25%, about 26%, about 27%, about 28%, about 29%, or about 30%, and therebetween.

Other suitable components for incorporation into the multi-phasic core material include antioxidants exemplified by citric acid, butylated hydroxytoluene (BHT), ascorbic acid, glutathione, retinol, a-tocopherol, β-carotene, a-carotene, ubiquinone, butylated hydroxyanisole, ethyl enediaminetetraacetic acid, selenium, zinc, lignan, uric acid, lipoic acid, N-acetylcysteine, and the like. An antioxidant, if present, is present in the compositions described herein in a total amount selected from the range of about 0.025% to about 1.0% by weight.

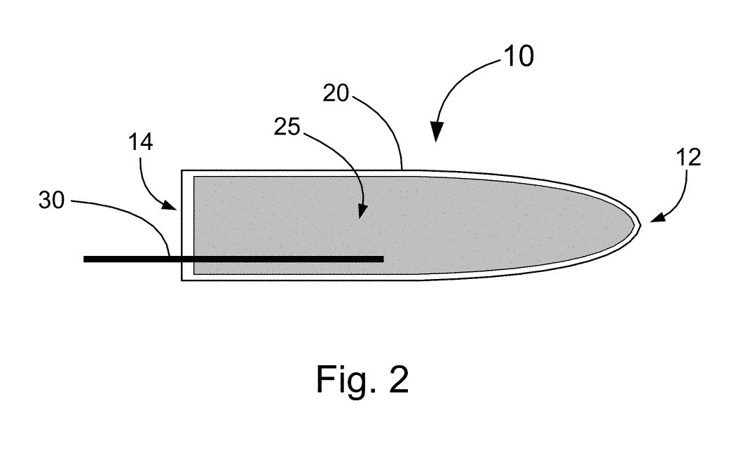

It is optional to insert an applicator stick 30 into the multi-phasic core material 25 of a nasal plug 10 as it is solidifying in a mold so that about 0.5 cm to about 1.5 cm of the applicator stick extends outward from the proximal end of the nasal plug (FIG. 2). The applicator stick 30 may be a wooden stick or a plastic stick or a polymeric stick, and the like. Alternatively, a length of a flexible material exemplified by a string, a cord, and the like, may be inserted into the multi-phasic core material of a nasal plug as it is solidifying in a mold so that a portion of about 0.5 cm to about 1.5 cm of the flexible material extends outward from the proximal end of the nasal plug. It is optional to place one end of a flexible material into one core material of a nasal plug as it is solidifying in a cavity of a mold and the other end of the flexible material into an adjacent core material in and adjacent cavity of the mold so that after the multi-phasic core material has solidified, two nose plugs are joined together by the flexible material that extends into both plug cores. Once the nosebleed has stopped, the nose plug can be removed via pulling on the applicator stick or the flexible material.

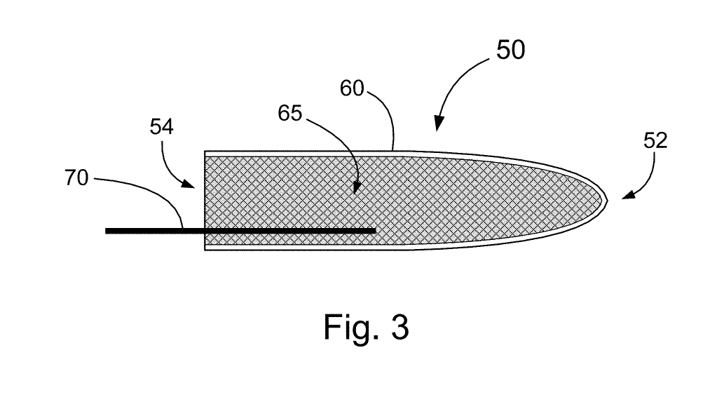

Another embodiment of a nose plug 50 according to the present disclosure is shown in FIG. 3 and has core 65 comprising a porous resiliently compressible substrate 65 into which has been imbedded an applicator stick 70 that extends outward from the distal end 54 of the nose plug 50. The porous resiliently compressible core material 65 is coated with a selected hemostatic compound 60.

Suitable hemostatic compounds include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof.

Suitable porous resiliently compressible substrates include materials that can be formed to bullet shapes or torpedo shapes or elliptical shapes or cylindrical shapes, for example man-made polymeric sponges, naturally occurring sponges, fluffy cellulosic materials, cotton, rolled gauze, the like, and mixtures thereof. The porous core substrate 65 is coated with a hemostatic composition 60. Suitable hemostatic compositions include but are not limited to ferrous sulfate, ferric sulfate, aluminum sulfate, ammonium sulfate, aluminum potassium sulfate, aluminum chloride, tannic acid, zinc chloride, the like, and mixtures thereof. Particularly suitable hemostatic compounds are ferrous sulfate, ferric sulfate, and mixtures thereof. It is optional if so desired, to infiltrate the porous resiliently compressible substrate 65 with a multi-phasic core material prepared as previously described in reference to FIGS. 1A and 1B.

If so desired, the applicator stick 70 may be substituted for with a length of a flexible material exemplified by a string, a cord, and the like. It is optional to place one end of a flexible material into one porous resiliently compressible core material 65 of a nasal plug 50 and the other end of the flexible material into an adjacent porous resiliently compressible core material 65 so that after the porous resiliently compressible core materials 65 have been coated with a hemostatic composition 60, two nose plugs 50 are joined together by the flexible material that extends into both plug cores.

The resiliently compressible nature of the porous substrate 65 makes it deformable as the present nose plug is inserted into a nostril so that a large portion of the outer covering of the nose plug 50 is in full contact with the nasal membranes lining the nostril. Once the nosebleed has stopped, the nose plug 50 can be removed via pulling on the applicator stick 70 or the flexible material.

Suitable packaging for the nose plugs disclosed herein include blister packages containing multiple numbers of the present nose plugs wherein each nose plug is sealed within a single blister. Such blister packages may contain one nose plug or two nose plugs or three nose plugs or four nose plugs or five nose plugs or six nose plugs or seven nose plugs or eight nose plugs or more. Such blister packages may then be sealed into a cardboard container, for example one blister package per container or two blister packages per container or three blister packages per container or four blister packages per container or more. Alternatively, multiples of the present nose plugs may be sealed in plastic bags that are then sealed in a cardboard container. Alternatively, multiples of the present nose plugs may be sealed into a plastic bottle or composite bottle or pressed fiber bottle. The multiples of the present nose plugs may be sealed in plastic bags before being sealed in the bottles. It is optional if so desired, to sterilize the sealed and packed nose plugs prior to distribution, for example by gamma ray irradiation.

While the embodiments of the present disclosure generally pertain to nose plugs for use by human subjects, the nose plugs described herein can be easily modified for use in stopping nosebleeds in other animals. For example, a number of surveys have shown that between 40% to 75% of horses have blood in their nostrils after strenuous activity. This is commonly referred to as exercise -induced pulmonary hemorrhage and frequently occurs in racing horses including among others, standardbreds, thoroughbreds, Arabians, appaloosas, quarter horses, and the like. Currently, the only treatment for nosebleeds in race horses is use of diuretics. But, there are no immediate solutions available to stop nosebleeds in horses.

Another embodiment of the present disclosure pertains to nose plugs for insertion into equine nostrils to stop nosebleeds. Equine nose plugs would have a similar configuration and composition as the nose plugs described previously in reference to FIGS. 1-3, except that the size of the plugs would be proportionally larger for equine applications. It would also be suitable to have the proximal out-facing end of the equine nose plugs to be flared out to provide broader surface coverage at the entrance to the nostril. Suitable diameters for equine nose plugs are in the range of about 2 cm, about 3 cm, about 4 cm, about 5 cm, about 6 cm and therebetween. Suitable lengths for equine nose plugs are in the range of about 5 cm, about 7.5 cm, about 10 c, about 12.5 cm and therebetween.

1. A deformable hemostatic nose plug, comprising:
a deformable core material; and
an outer coating provided on said deformable core material, said outer coating comprising a hemostatic compound,
wherein the nose plug has a tapered distal end for insertion into a nostril and a proximal end whereby the nose plug is inserted into the nostril.

2. The deformable nose plug according to claim 1, wherein the hemostatic compound is one of ferrous sulfate, ferric sulfate, aluminum sulfate, ammonium sulfate, aluminum potassium sulfate, aluminum chloride, tannic acid, zinc chloride, and mixtures thereof.

3. The deformable nose plug according to claim 2, wherein the hemostatic compound is one of ferrous sulfate, ferric sulfate, and mixtures thereof.

4. The deformable nose plug according to claim 1, wherein the deformable core material comprises a multi-phasic core material wherein a first phase of the multi-phasic core material is characterized by a solid or a semi-solid structure at a temperature of 35° C. or less, and wherein a second phase of the multi-phasic core material is characterized by liquifaction at a temperature of 37° C. or greater.

5. The deformable nose plug according to claim 4, wherein the deformable core material additionally comprises one or more of a clotting factor, an antibiotic composition, an antimicrobial agent, an antihistamine, a decongestant, an antioxidant, an emollient, and combinations thereof.

6. A deformable nose plug according to claim 1, wherein the deformable core material comprises a porous resiliently compressible substrate.

7. A deformable nose plug according to claim 6, wherein the porous resiliently compressible substrate is one of polymeric sponges, naturally occurring sponges, fluffy cellulosic materials, cotton, rolled gauze, and mixtures thereof.

8. The deformable nose plug according to claim 7, wherein the deformable core material additionally comprises one or more of a clotting factor, an antibiotic composition, an antimicrobial agent, an antihistamine, a decongestant, an antioxidant, an emollient, and combinations thereof.

9. The deformable nose plug according to claim 1, additionally having an applicator embedded into the deformable core material, said applicator extending outward from the proximal end of the nose plug.
